(19) United States
(12) Patent Application Publication
(10) Pub. No.: US 2008/0274501 A1
(43) Pub. Date: Nov. 6, 2008

(54) METHOD OF PURIFYING ACIDIC PROTEINS EXPRESSED IN PLANTS

(76) Inventors: Chenming ZHANG, Blacksburg, VA (US); Chris Holler, Lexington, KY (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 12/114,507

(22) Filed: May 2, 2008

Related U.S. Application Data
(60) Provisional application No. 60/915,457, filed on May 2, 2007.

(51) Int. Cl. C12P 21/00; C07K 14/00

(52) U.S. Cl. 435/69.1; 530/412; 530/421; 530/417

(57) ABSTRACT

The present invention provides a rapid and relatively simple process for purification of acidic proteins expressed in tobacco cells. The process comprises three main purification steps: precipitation with polyethyleneimine, column chromatography with a hydrophobic interaction resin, and column chromatography with hydroxyapatite. The process provides pure or essentially pure protein at a very high yield.

### CROSS-REFERENCE TO RELATED APPLICATIONS

This application relies on and claims the benefit of the filing date of U.S. provisional patent application No. 60/915,457, filed 2 May 2007, the entire disclosure of which is incorporated herein by reference.

### BACKGROUND OF THE INVENTION

1. Field of the Invention

The present invention relates to the fields of molecular biology and protein biochemistry. More specifically, the invention relates to purification of proteins, typically recombinantly expressed, from tobacco cells.

2. Description of Related Art

Expression of recombinant therapeutic proteins in transgenic plants can have a tremendous impact on the biopharmaceutical industry and crop production around the world. Numerous recombinant proteins, including antibodies, vaccines, hormones, and growth regulators have already been expressed in crops such as corn, rice, soybean, and tobacco, among many others. Tobacco, in particular, is an attractive host for the commercial production of recombinant therapeutic proteins (Stoger et al., 2002a; Stoger et al., 2002b). It is a non-food and non-feed crop, so the threat of contamination of a food or feed supply is minimized. Furthermore, an abundance of biomass can be produced by simply planting more transgenic plants if the protein is expressed in the leaf tissue (Daniell et al., 2001). As with all plants, tobacco does not harbor human pathogens, which provides a safer alternative to mammalian or bacterial production systems.

Even if recombinant therapeutic proteins can be expressed efficiently in tobacco, there is still a great challenge in developing tobacco as an effective and economical host for producing these proteins on a large scale commercial basis. Because therapeutic proteins expressed in tobacco can not be delivered orally, the protein must be purified to high quantities and purity for use in clinical trials. Downstream processing, which includes extraction, purification, and characterization of the target protein, is estimated to account for 80% or more of the overall production cost (Kusnadi et al., 1998b). In addition, detailed protein purification methods and cost analyses are not well studied in tobacco, or in other leafy crops (Nikolov and Woodard, 2004).

An aqueous extract obtained from tobacco vegetative tissue is a complex mixture of native plant proteins, carbohydrates, nucleic acids, and other impurities, such as phenolics and alkaloids that must be separated from the target protein. It is estimated that the overall nature of native tobacco proteins is acidic in nature (Balasubramaniam et al., 2003). The overall acidity is largely attributed to the presence of the chloroplast storage protein, ribulose 1,5 bisphosphate carboxylase-oxygenase (Rubisco). Rubisco is found in most vegetative tissue and is the most abundant protein in the world. In tobacco, Rubisco may account for more than 25% of the total protein and 50% of the total chloroplast protein (Garger et al., 2000; Shewry and Fido, 1996). Rubisco is a hexadecameric protein with eight large subunits (average MW 50-60 kD, pI 6.0) and eight small subunits (average MW 12-20 kD, pI 5.3) (Menkhaus et al., 2004; Shewry and Fido, 1996). Thus, purification of an acidic recombinant protein from tobacco may be more challenging than purification of a basic protein (Balasubramaniam et al., 2003). Nevertheless, there are numerous acidic therapeutic proteins that have been expressed in tobacco that would greatly benefit from efficient purification processes.

In addition to native plant proteins, nucleic acids, and toxic alkaloids such as nicotine, the presence of phenolics in a tobacco extract could be problematic for protein purification. Phenolics can form complexes with proteins or interfere with adsorption processes such as column chromatography (Cheryan, 1980; Jervis and Pierpoint, 1989; Kusnadi et al., 1998a). However, inclusion of a reducing agent such as beta-mercaptoethanol (BME) or dithiothreitol (DTT) as well as a phenolic binding agent such as polyvinyl polypyrrolidone (PVPP) during the protein extraction and recovery process is believed to alleviate many of these interferences (Holler et al., 2007). Incorporating a scalable non-chromatographic step early in a purification scheme may be advantageous for purifying recombinant proteins from large amounts of transgenic tobacco (Menkhaus et al., 2004). Application of a crude tobacco extract to a chromatography column is not feasible due to column fouling and plugging overextended use. Polyelectrolyte precipitation with polyethyleneimine (PEI) has shown to be a promising application for removing large amounts of native tobacco impurities as well as providing high recovery and concentration of an acidic recombinant protein in preparation for chromatography steps (Holler et al., 2007).

Numerous methods for purifying recombinant therapeutic proteins from transgenic tobacco rely heavily on molecular modification of the protein or affinity steps (Desai et al., 2002; Lige et al., 1998; Mejare et al., 1998; Sehnke and Ferl, 1999). However, these processes are costly due to their high specificity and may not be feasible for large scale commercial production of therapeutic proteins. Therefore, it may be beneficial to develop purification schemes that utilize more general chromatography steps, such as ion-exchange, hydrophobic interaction, and hydroxyapatite that can be used to separate a wide range of proteins.

### SUMMARY OF THE INVENTION

The present invention addresses needs in the art by providing a purification scheme for purification of acidic proteins that are expressed in plant cells, such as those of leafy crops (e.g., lettuce, alfalfa, tobacco). The purification scheme is equally applicable to proteins that are naturally produced in plants and those that are recombinantly produced. The purification scheme greatly enhances the ability to produce and purify proteins of interest to the biopharmaceutical and research industries, and provides a new means for recombinantly expressing and efficiently purifying proteins in a eukaryotic system.

In one aspect, the invention provides a method or process for purifying or isolating a protein that is expressed in one or more plant cells, such as those expressed in transgenic tobacco plants. In general, the method comprises: expressing a protein in at least one plant (e.g., tobacco) cell; lysing the cell(s); and purifying the protein through a method comprising: polyelectrolyte precipitation, hydrophobic interaction chromatography, and hydroxyapatite chromatography. Purification or isolation can be to any extent, but is preferably to a point where no contaminating proteinaceous substances are detectable. While not limited to a particular type of protein, typically, proteins purified according to the method of the present invention are acidic and are recombinantly produced in the cell(s). The method can further comprise a centrifugation step or equivalent step to remove solid or insoluble materials from soluble materials in the cell lysate. The method may also further comprise one or more concentration steps to concentrate the protein, for example by way of salt precipitation or ion exchange chromatography on a high-binding capacity, low bed volume chromatography column and ultrafiltration/diafiltration.

In another aspect, the invention provides a method of obtaining recombinant proteins from plant cells (e.g., tobacco plant cells). In general, the method comprises: creating a transgenic plant cell; expressing a recombinant protein in the cell; and obtaining the recombinant protein from the cell using a combination of polyelectrolyte precipitation, hydrophobic interaction chromatography, and hydroxyapatite chromatography. Preferably, the cell comprises a part of a transgenic plant, such as a transgenic tobacco plant. While not so limited, the recombinant protein is preferably an acidic protein.

The methods of the invention can be high-yield methods, resulting in a yield of at least 30% of original protein of interest. For example, the methods may be practiced to achieve a yield of recombinantly produced protein of 35%, 40%, or more. The methods rely on relatively few purification steps, and thus reduce the number of points where target protein can be lost. In addition, the relative simplicity of the purification scheme makes the scheme widely applicable and useful for purification of acidic proteins in general. Likewise, the relative simplicity of the purification scheme makes the scheme widely applicable and useful for purification of proteins from numerous plants. Non-limiting examples of proteins for which the present purification methods may be applied are presented in Table 1.

TABLE 1
Examples of Therapeutically Relevant Proteins

| | | MW | | | |
|---|---|---|---|---|---|
| | Potential | (kD) | | | |
| Protein | application | subunit | pl | Expression Level** | Source(s) |
| Human protein C | Protein C | 62^{a} | 4.4-4.8^{b} | <0.01% TSP^{c} | (Kisiel and Davie 1981)^{a} |
| (serum protease) | pathway | | | | (Discipio and Davie 1979)^{b} |
| | | | | | (Cramer et al. 1999)^{c} |
| Norwalk capsid protein | Norwalk | 58 | 5.25-5.5* | 0.23% TSP | (Mason et al. 1996) |
| | virus | | | | |
| Angiotensin-I-converting | hypertension | 1.38* | 6.00* | 100 μg/g of | (Hamamoto et al. 1993) |
| enzyme inhibitor (coat | | | | fresh tissue | |
| protein-ACEI complex) | | | | | |
| binding subunit of E. coli | cholera and | 11.6 | 5.05* | 0.001% of soluble | (Haq et al. 1995) |
| heat-labile enterotoxin | E. coli | | | leaf protein | |
| (Lt-B) | diarrhea | | | | |
| Human serum albumin | Liver | 67-69 | 5.85* | 0.02% TSP (nuclear)^{d} | (Siimons et al. 1990)^{d} |
| | cirrhosis | | | 11.1% (chloroplast)^{e} | (Fernandez-San Millan et al. 2003)^{e} |
| c-Myc | Cancer | 49* | 5.33* | Not reported | (Beachy et al. 1996) |
| Human granulocyte- | Neutropenia | 14.5* | 5.21* | Not reported | (Ganz et al. 1996) |
| macriphage colony- | | | | | (Goddiin and Pen 1995) |
| stimulating factor | | | | | |

*estimated using ExPASy calculator
**TSP, total soluble protein

In yet another aspect, the invention provides isolated or purified proteins expressed in plant cells, such as tobacco plant cells. While not so limited, preferably the proteins are acidic proteins that are purified by way of a method of the present invention. Typically, the proteins are recombinant proteins that are expressed or overexpressed in transgenic plants.

### BRIEF DESCRIPTION OF THE DRAWINGS

The accompanying drawings, which are incorporated in and constitute a part of this specification, illustrate several embodiments and features of the invention, and together with the written description, serve to explain certain principles of the invention.

FIG. 1 depicts a diagram of a processing scheme for the purification of an acidic recombinant protein from transgenic tobacco leaf tissue.

FIG. 2 shows a representative chromatogram of a hydrophobic interaction chromatography (HIC) step used as a step in the purification of rGUS from transgenic tobacco.

FIG. 3 shows a hydroxyapatite chromatography (HAC) scheme used as a step in the purification of rGUS from transgenic tobacco after PEI precipitation and HIC.

FIG. 4A shows a hydroxyapatite chromatography scheme showing increased separation of rGUS from native tobacco proteins with 16 pooled fractions.

FIG. 4B shows the results of an SDS-PAGE of the corresponding individual fractions collected and concentrated using Microcon centrifugal concentration devices.

FIG. 5A shows the results of SDS-PAGE of the entire purification scheme stained with Bio-Safe Coomassie stain.

FIG. 5B shows silver staining of the gel from FIG. 5A.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is provided to give the reader a better understanding of various details and features of the invention, and should not be understood as a limitation on the scope or content of the invention.

Tobacco has proven to be a promising alternative for the production of recombinant therapeutic proteins and offers numerous advantages over other plants as a host system. However, the recovery and purification steps needed to obtain a protein at high recovery and purity have not been well investigated. According to the present invention, a process was developed to purify proteins from tobacco plant cells. Specifically, an exemplary embodiment provides a purification process for a model acidic protein, recombinant beta-glucuronidase (rGUS), from transgenic tobacco leaf tissue. The exemplary method comprises three main steps after extraction of the recombinant protein from the recombinant cells: polyelectrolyte precipitation, hydrophobic interaction chromatography (HIC), and hydroxyapatite chromatography (HAC). Poylelectrolyte precipitation can be performed with any substance having the appropriate chemical characteristics, as exemplified by polyethyleneimine. Selection of an optimal polyelectrolyte is a straightforward matter that is well within the routine abilities of those of skill in the art. Purification with hydrophobic interaction chromatography can be performed with any of a number of suitable hydrophobic interaction resins, as known in the art. Selection of an optimal resin will be a simple task that is straightforward and routine.

Using this three-step process, up to 40% or more of the initial rGUS activity could be recovered to near homogeneity as judged by SDS-PAGE. This work demonstrates that acidic recombinant proteins expressed in tobacco may be purified to high yield with high purity in a minimal amount of steps that are suitable for scale-up. Furthermore, the general steps used in this process may suggest that a wide variety of acidic proteins, and particularly those that are expressed at high levels, such as recombinant proteins, may be purified in a similar manner from transgenic tobacco or other leafy crops.

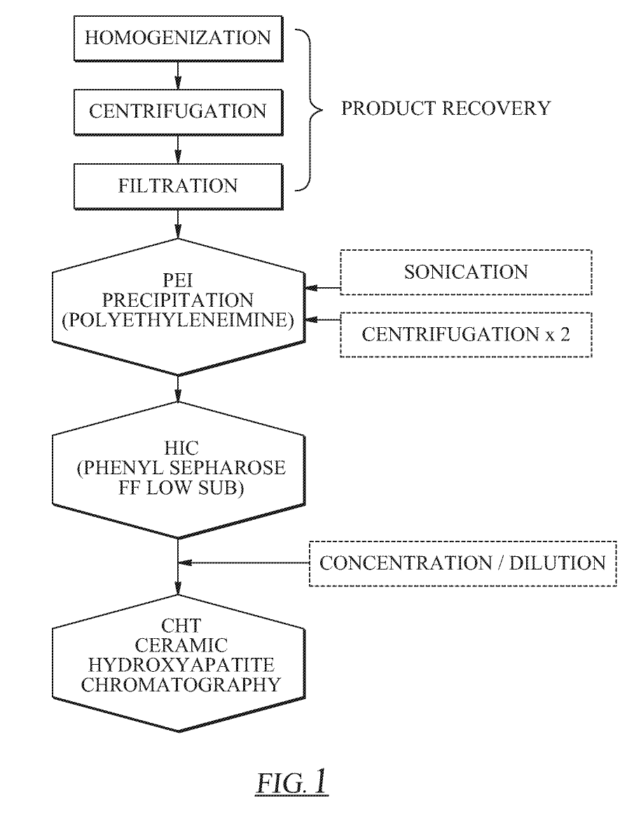

The invention provides a general and scalable process for the purification of proteins from tobacco cells. Non-limiting examples relate to the model acidic recombinant protein, rGUS, from transgenic tobacco leaf tissue. A purification scheme for rGUS is depicted in FIG. 1. The process outlines the extraction and recovery of rGUS into a crude aqueous tobacco extract followed by a purification process that has three major steps: polyelectrolyte precipitation, hydrophobic interaction chromatography, and hydroxyapatite chromatography. More specifically, FIG. 1 shows a diagram of a downstream processing scheme for the purification of an acidic recombinant protein from transgenic tobacco leaf tissue according to the present invention. The model protein studied was recombinant beta-glucuronidase and the steps chosen were kept to the minimal amount needed to provide high recovery and purity of the target protein. In addition, the steps were kept general for the application to a wide range of acidic recombinant proteins expressed in tobacco or other leafy crops. Although an additional sample concentration step was used after hydroxyapatite chromatography to visualize the protein bands on SDS-PAGE, this step is not necessarily included as part of the overall purification scheme.

### EXAMPLES

The invention will be further explained by the following Examples, which are intended to be purely exemplary of the invention, and should not be considered as limiting the invention in any way.

The following Examples show that an acidic recombinant protein (rGUS) can be efficiently purified from transgenic tobacco to high yield and purity in just three main steps after the initial extraction. A polyethyleneimine (PEI) precipitation step served as an effective non-chromatographic step for initial fractionation and concentration of the target protein. A hydrophobic interaction chromatography (HIC) step served the purpose of removing impurities such as PEI and nucleic acids obtained in the first step. The hydroxyapatite chromatography (HA) step served as the “polishing” step where rGUS was effectively separated from the remaining native tobacco proteins, most notably Rubisco. The protocol resulted in an approximately 40% product yield (based on the initial rGUS activity) after the final step, excluding the final concentration step needed for band visualization on SDS-PAGE. The rGUS was recovered as a purified protein to near homogeneity as judged by Coomassie and silver stained SDS-PAGE. These results show that acidic recombinant proteins can be efficiently purified from a transgenic tobacco extract in a minimum number of general steps. All of the steps incorporated in the purification scheme have the potential to be scaled up for large-scale protein production.

Unless otherwise indicated, the following materials and general methods were used in the following examples:

Transgenic tobacco growth and protein extraction: Transgenic tobacco plants were grown as described previously (Holler et al., 2007). Fresh tobacco leaves were excised from a plant each time an extract was prepared and the tissue was weighed out and placed in a 50 mL conical tube. The extraction buffer contained 50 mM sodium phosphate (NaPi), pH 7; 10 mM BME; and 1 mM EDTA. All extractions were carried out at a ratio of 1:5 (w/v) with ice cold buffer (around 4° C.). The resulting sample was homogenized until no large particulate material remained (approximately one minute). Immediately after homogenization, the sample was decanted to another tube containing a 2% (w/v) pre-hydrated PVPP solution. The extract was then vortexed vigorously and allowed to set at room temperature for 15 minutes. After centrifugation at 4° C. and 17,003×g for 20 minutes, the supernatant was removed and filtered through a 0.22 micrometer (um) syringe filter.

Polyelectrolyte precipitation: Polyethyleneimine (PEI; long chain average MW 750 kD; 50% (w/v) aqueous solution from Sigma) was diluted to 10 mg/mL in deionized (DI) water and adjusted to pH 7 with concentrated HCl. This solution was added to 7 mL extract at a dosage of 800 mg PEI/g total protein. The necessary amounts of DI water and PEI stock were added to bring the final volume up to 8.4 mL. After addition of the polyelectrolyte, the samples were vortexed vigorously for 10 seconds and then allowed to precipitate at room temperature for 30 minutes. Samples were then centrifuged for 20 minutes at 17,003×g at room temperature (approx. 22-25° C.). The supernatant was removed and saved for later analysis. The pellets were washed with 1 mL of DI water and then 1.5 mL of resuspension buffer (50 mM NaPi, pH 7.0; 10 mM BME; 1 mM EDTA; and 0.5 M NaCl) was added. Because the pellets could not be resuspended by simply vortexing, the samples were sonicated (Fisher Sonic Dismembrator, Model 500) for five seconds and then re-centrifuged for 10 minutes at 17,003×g to re-pellet the unwanted debris. The supernatant was removed from these samples and centrifuged again at 16,400×g for 10 minutes (Marathon 16 KM, Fisher Scientific). The supernatant was removed from these samples and assayed as the “pellet” data.

FPLC chromatogaphy: Chromatography experiments were performed using an AKTA Explorer 100 (GE Healthcare, Uppsala, Sweden) fast-performance liquid chromatography (FPLC) system controlled by the Unicorn software. Phenyl Sepharose 6 Fast Flow (low substitution) was purchased from GE Healthcare and used for hydrophobic interaction chromatography. An HR 5/10 glass column (GE Healthcare), 10 cm×5 cm (i.d.), was packed to a bed height of approximately 5.1 cm (1 mL bed volume). The equilibrating buffer was 50 mM NaPi, pH 7.0; and 1.5 M ammonium sulfate (AS) (referred to as Buffer A1). Proteins were eluted with a linear gradient of 50 mM NaPi, pH 7.0 (referred to as Buffer B1) from 0% to 100% over 10 minutes. All flow rates used were 1 mL/minute and fractions were collected in 2 mL aliquots.

CHT ceramic hydroxyapatite (Macro-prep Type I; 80 um) was purchased from Bio-Rad Laboratories (Hercules, Calif., USA). The resin was resuspended in 200 mM sodium phosphate buffer, pH 9 and packed as a slurry in a Tricorn column (GE Healthcare), 10 cm×1 cm (i.d.), to a bed height of approximately 10.2 cm (8 mL bed volume). The equilibrating buffer was 10 mM NaPi, pH 6.8 (referred to as Buffer A2). Proteins were eluted over a linear gradient with 400 mM NaPi, pH 6.8 (referred to as Buffer B2) as the elution buffer. All flow rates used were 1 mL/minute and fractions were collected in 1 mL aliquots. Collected fractions were pooled and concentrated when needed in either Microcon (YM-10) or Amicon Ultra (5k) centrifugal filter devices purchased from Millipore (Bedford, Mass., USA).

Analytical methods: Protein concentration was determined by Bio-Rad assay (microtiter procedure) with bovine serum albumin (BSA) as the standard. All assays were carried out in Greiner 96-well, clear, flat bottom microtiter plates from USA Scientific (Ocala, Fla., USA) and performed in duplicates. Absorbance measurements were read at 595 nm on a Bio-Tek Synergy microplate reader (Bio-Tek Instruments, Inc., Winooski, VT, USA). GUS activity was measured as reported previously (Holler et al., 2007). This assay utilizes the ability of GUS to hydrolyze p-nitrophenyl beta-D-glucuronide (PNPG) to release chromophore p-nitrophenol (PNP), and GUS activity is reported in units of activity (U)/mL (Jefferson and Wilson, 1991). One unit of GUS activity was defined as the amount needed to liberate one nanomole (nmol) of PNP/minute at room temperature and pH 7.0. Kinetic absorbance measurements were read at room temperature and 405 nm every 50 seconds for a total of eight minutes on a microplate reader.

SDS-PAGE samples were reduced with dithiothreitol (DTT) and run on 4-12% Novex Bis-Tris mini gels with MOPS as the running buffer obtained from Invitrogen (Carlsbad, Calif., USA). After running, the gels were washed with DI water and bands were visualized using Bio-Safe Coomassie stain (Bio-Rad) or SilverQuest staining kit (Invitrogen). The gels were scanned with a Bio-Rad ChemiDoc XRS imager and analyzed using Quantity One Software.

### EXAMPLE 1

### Protein Extraction From Transgenic Ttobacco

The product recovery step is one of the most important steps in downstream processing as it will dictate how much initial protein is present for purification. The aim of the recovery step is to maximize target protein extraction into an aqueous medium while minimizing protein degradation. For many studies, an extract can be obtained by grinding frozen leaf tissue under liquid nitrogen followed by addition of an appropriate extraction buffer. However, this process is not feasible for large-scale extraction on large amounts of tobacco leaf biomass. Therefore, in the method of this embodiment of the invention, extraction was accomplished by homogenization, which sheared the leaf tissue in an aqueous buffer. During this stage, there are several important factors to consider for minimizing protein degradation. First, 10 mM BME was added to the buffer prior to homogenization to keep the environment in a reduced state, preventing harmful oxidation that might alter the foreign protein's structure and render it inactive. Second, the buffer was kept at ice-cold temperature prior to homogenization to minimize proteolysis or protein denaturing due to increased temperature during the homogenization process. Third, immediately after homogenization, pre-hydrated PVPP was added to the solution. The PVPP serves to bind free phenolics that might otherwise form complexes with proteins or foul chromatographic columns later used in the purification process. This was found to be a highly advantageous step in tobacco protein extraction as tobacco contains extremely high amounts of phenolics, up to 30 mg/g dry weight (Davis and Nielson, 1999). Because PVPP is insoluble, it is easily removed in the pellet during a centrifugation step. After centrifugation, the remaining fine particulates are removed via filtration through a 0.22-um syringe filter. Over nine independent experiments, the average starting total protein in a tobacco extract was 1.37±0.54 mg/mL and the average initial rGUS activity was 128.2±33.1 U/mL.

In the experiments conducted for this study, rGUS extraction was not optimized, but several different combinations of extraction buffers were investigated before a simple three component system was chosen. The combination of low salt (50 mM phosphate), a reducing agent (10 mM BME), and an anti-chelating agent (1 mM EDTA) was found to consistently provide high amounts of initial rGUS activity. Additional components, such as protease inhibitors (phenylmethylsulphonyl fluoride, PMSF) or detergents (Triton X-100, Tween 20, Sarcosyl) might be advantageous for some extraction procedures to minimize protease degradation or disrupt cellular membranes respectively, but these additives should be removed during the purification process.

### EXAMPLE 2

### PEI Precipitation

After a transgenic tobacco extract was obtained, the first main step in the purification process was polyelectrolyte precipitation. Polyethyleneimine was added at a dosage of 800 mg PEI per gram total protein to ensure near complete precipitation of rGUS and maximum recovery in the pellet fraction, as reported previously (Holler et al., 2007). While a dosage of 800 mg PEI per gram total protein results in near complete precipitation of rGUS, a number of other native tobacco proteins co-precipitate, most notably the acidic chloroplast storage protein, ribulose 1,5-bisphosphate carboxylase-oxygenase (Rubisco). Increasing the PEI dosage effectively increases the amount of Rubisco co-precipitated with rGUS, leading to modest enrichment values (Holler et al., 2007). Previously it was reported that sonication and subsequent centrifugation of the pellet was necessary after the addition of resuspension buffer to adequately recover rGUS from the precipitated pellet (Holler et al., 2007). An additional step of centrifugation was performed here in order to remove fine particulates before chromatography runs. This step did not significantly affect rGUS recovery or enrichment values of the precipitation step.

For the experiments reported here, the PEI precipitation data are summed up in Table 2. The PEI precipitation step was a useful step in the purification process because it removed large amounts of impurities (approximately 65% native tobacco proteins). This removal should help improve column life and performance in later chromatography steps. Also, more than 95% of the target protein was recovered, on average, and the sample volume was greatly reduced (for example, from 8.4 mL to 1.5 mL for a 5.6 x concentration) before chromatographic separation.

TABLE 2
PEI Precipitation Data (total of 9 experiments)

| | Total Protein | % total protein | | rGUS |
|---|---|---|---|---|
| | (mg/mL) | recovered in pellet | Enrichment | Recovery |
| Average | 1.37 | 36.05% | 2.72 | 96.41% |
| Standard | 0.54 | 5.64% | 0.47 | 6.42% |
| Deviation | | | | |

### EXAMPLE 3

### HIC Optimization

Hydrophobic interaction chromatography (HIC) was carried out as the second main step in the purification scheme. After PEI precipitation, the sample (1.5 mL) was applied directly to the HIC column with no additional salt added. The sample was loaded onto the column followed by an additional 2 mL of equilibration buffer (A1) on top of the sample, which ensured sufficient salt for binding. Our previous results suggest that an HIC step after PEI precipitation could not efficiently separate rGUS from many native tobacco proteins (e.g., Rubisco), leading to an enrichment ratio of approximately 6.55 with only 53.5% recovery (Holler et al., 2007). Several other HIC resins were investigated, including Phenyl Sepharose FF (high substitution), Octyl Sepharose FF, and Butyl Sepharose FF, but separation was not achieved and recoveries were often lower than with Phenyl Sepharose FF (low substitution) (data not shown). Therefore, it was concluded that HIC chromatography would not be able to provide adequate separation to produce a highly pure rGUS.

The HIC step, however, was found to serve a valuable purpose in the purification scheme. Several fractions collected during the flow-through were a light brownish color with extreme cloudiness observed upon diluting with low salt buffer. This suggests that both color-containing pigments (chlorophyll) and PEI are removed during this process. In addition, it is believed that a large amount of nucleic acids are also removed, either bound to PEI or in isolated form, due to the fact that a large peak was observed at 215 nm on the chromatogram during the flow-through. Other methods were attempted to remove PEI and nucleic acids including de-salting column and dialysis; however, in both cases precipitation of the proteins occurred and rGUS activity could not be recorded (data not shown). As a result, the HIC step also serves as the only viable de-salting process needed for the next step in the purification process.

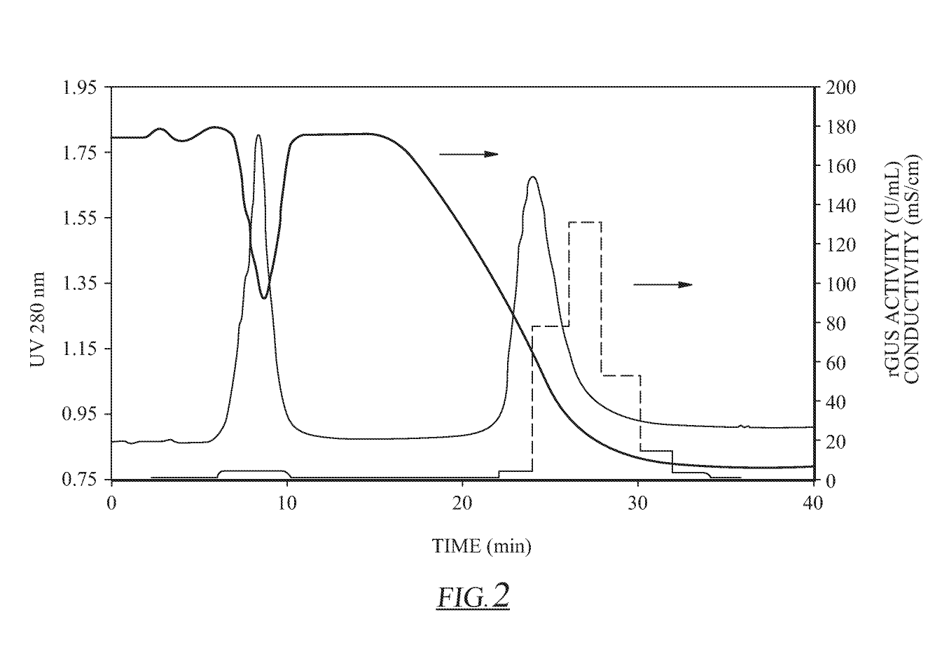

The HIC step in this process was thus optimized for maximum rGUS recovery with little regard for additional purification (enrichment) after PEI precipitation (FIG. 2). More specifically, FIG. 2 shows a representative chromatogram of the optimized HIC step used as the second step in the purification of rGUS from transgenic tobacco. A total of 1.5 mL sample was loaded to the column after PEI precipitation at 800 mg PEI per gram of total protein. The thick dashed line represents the rGUS activity collected in each of the fractions and the thin dotted line represents the elution gradient from 50 mM NaPi, pH 7.0 and 1.5 M ammonium sulfate to 50 mM NaPi, pH 7.0 and no ammonium sulfate.

The results of three identical HIC optimization trials are shown in Table 3. As can be seen, nearly 78% of the initial rGUS activity could be recovered after the HIC step (83.34% of the total activity loaded from the PEI step). The enrichment values were not calculated and the total volume collected from the HIC step was 10-12 mL (5-6 fractions).

TABLE 3
HIC Optimization Data

| | Trial | Trial | Trial | | |
|---|---|---|---|---|---|
| | 1 | 2 | 3 | Average | stdev |
| Starting total protein (mg/mL) | 0.73 | 0.92 | 0.65 | 0.77 | 0.14 |
| % rGUS activity recovered | 92.84 | 97.12 | 94.48 | 94.81 | 2.16 |
| after PEI precipitation | | | | | |
| Enrichment ratio after PEI | 2.61 | 2.60 | 3.1 | 2.8 | 0.28 |
| precipitation | | | | | |
| % rGUS activity recovered in | 4.37 | 9.03 | 3.03 | 5.48 | 3.15 |
| flow-through (from original) | | | | | |
| % rGUS activity recovered in | 77.00 | 80.86 | 76.07 | 77.98 | 2.54 |
| pooled fractions (from original) | | | | | |
| Total volume of pooled | 12 | 10 | 12 | | |
| fractions after HIC (mL) | | | | | |

### EXAMPLE 4

### Full Purification Process

It was anticipated that size-exclusion chromatography could be used as a third step to fully separate rGUS from Rubisco based on the size of the polymeric forms of both proteins which are 270 and 560 kDa, respectively (Holler et al., 2007). However, several runs using Sephacryl S-300 HR resin (XX 16/20 column, G.E. Healthcare) yielded little additional separation of Rubisco and rGUS after PEI precipitation and HIC, respectively (data not shown). Therefore, ceramic hydroxyapatite resin was investigated as an alternative chromatography step.

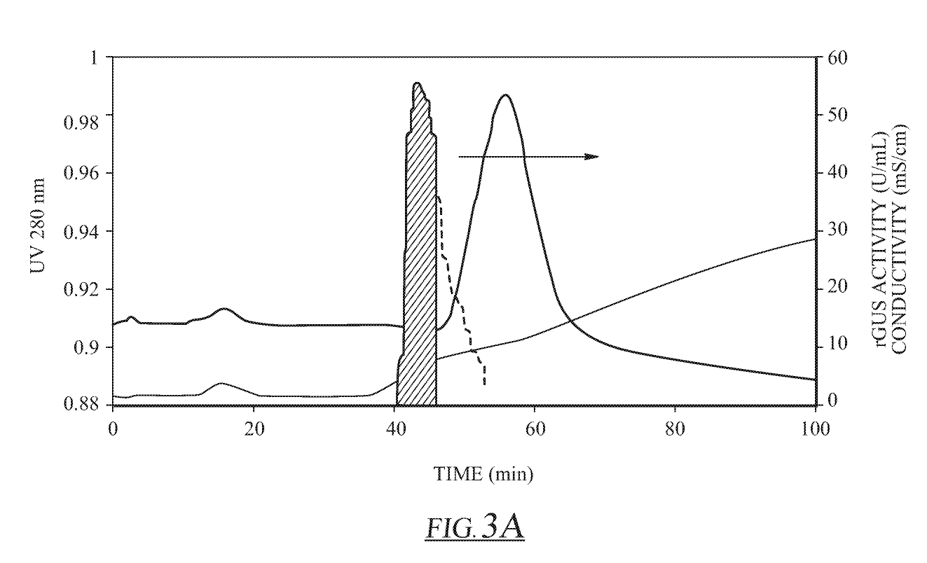

Hydroxyapatite (HA) can be used for the binding of acidic proteins and has the potential for scale-up. A low salt sample is typically required for the binding of proteins to the HA resin (10 mM NaPi). As up to 12 mL of sample was collected after the HIC step, it was desired to reduce the sample volume before loading to the HA column. Therefore, the samples were concentrated down to less than 1 mL with Amicon Ultra-15 centrifugal filter devices and then subsequently diluted with deionized water to lower the NaPi concentration close to 10 mM and the estimated ammonium sulfate concentration to less than 100 mM. The total volume loaded onto the HA column was approximately 3.5 mL. Preliminary experiments showed the possibility that rGUS may be separated from Rubisco and other native tobacco proteins, at least partially, on a hydroxyapatite (HA) column after subsequent steps of PEI precipitation and HIC (FIG. 3a). A simple linear elution gradient of 0% to 100% B2 over 80 minutes yielded between 40%-50% of the initial rGUS activity during the elution. The pooling of five fractions resulted in a homogeneous band on SDS-PAGE, which corresponded to nearly 25% of the initial rGUS activity (FIG. 3b).

More specifically, FIG. 3 shows the results of an HA scheme used as the third step in the purification of rGUS from transgenic tobacco after PEI precipitation and HIC. The chromatogram in Panel A shows the elution of rGUS (thick dashed line) prior to the elution of the main native tobacco proteins. Panel B shows a silver stained SDS-PAGE gel of the five pooled fractions (shaded in black), which showed a homogeneous band at 68 kD corresponding to rGUS. The gel was silver stained for maximum detection of protein bands.

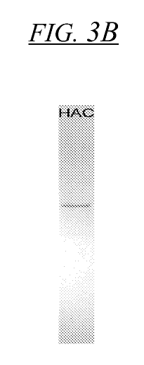

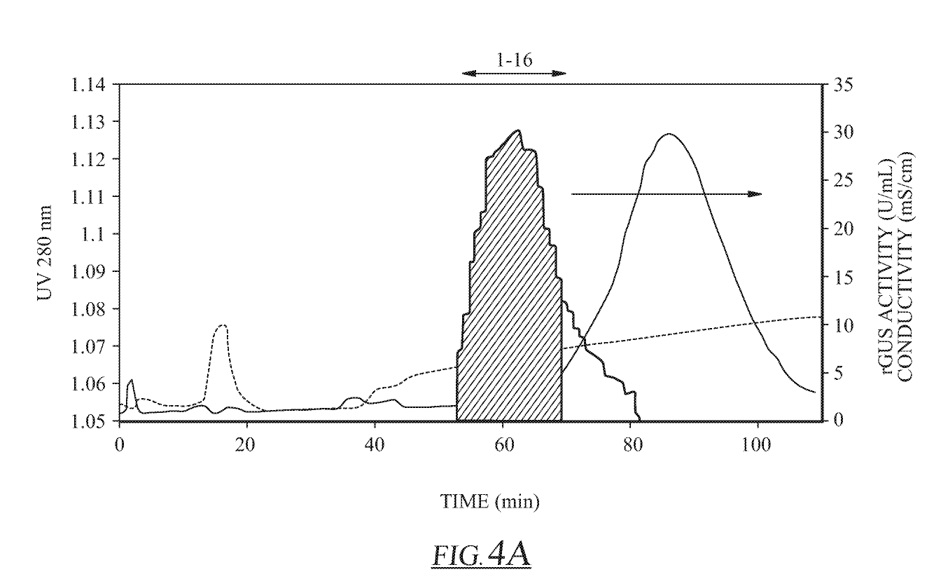

As shown in FIG. 3a, recombinant GUS begins to elute just prior to the native tobacco proteins during hydroxyapatite chromatography separation. Optimization by extended gradient over the salt concentration where proteins eluted yielded greatly improved results with respect to separation. More specifically, as shown in FIG. 4A, optimization of a hydroxyapatite chromatography scheme showed increased separation of rGUS from native tobacco proteins with the 16 pooled fractions shaded in black. FIG. 4B shows an SDS-PAGE result of the corresponding individual fractions collected and concentrated using Microcon centrifugal concentration devices. The lane marked (+) is a Sigma GUS standard used as a positive control.

For the optimized run, total rGUS activity recovered in all fractions was still 50%. A total of 16 fractions were pooled, which yielded 40% of the initial activity and were judged to be highly pure by SDS-PAGE (FIG. 4b). Therefore, this simple optimization procedure increased purified rGUS recovery significantly from 25% to 40%. Further optimization of this step may lend itself to recovering the maximum amount of rGUS as a pure fraction, up to 50% of the initial protein activity. The final enrichment ratio could not be calculated due to the fact that it is difficult to determine the accurate protein concentration in the sample. The results for the entire purification scheme are presented in Table 4.

TABLE 4
Data From Full Purification Scheme

| | | Final Vol. | | |
|---|---|---|---|---|
| Step | Tot. Act. (U) | (mL) | E.R. | Recovery |
| Extraction | 862.44 | 7 | n/a | 100.00% |
| PEI Precpitation | 808.45 | 1.5 | 3.10 | 93.74% |
| HIC fractions | 724.10 | 6 | 6.22 | 83.96% |
| (before concentration) | | | | |
| HIC fractions (after | 662.20 | 3.5 | 4.92 | 76.78% |
| concentration/dilution) | | | | |
| HAC fractions | 438.42 | 28 | n/a | 50.84% |
| (total rGUS) | | | | |
| HAC fractions | 349.66 | 16 | n/a | 40.54% |
| (purified rGUS) | | | | |

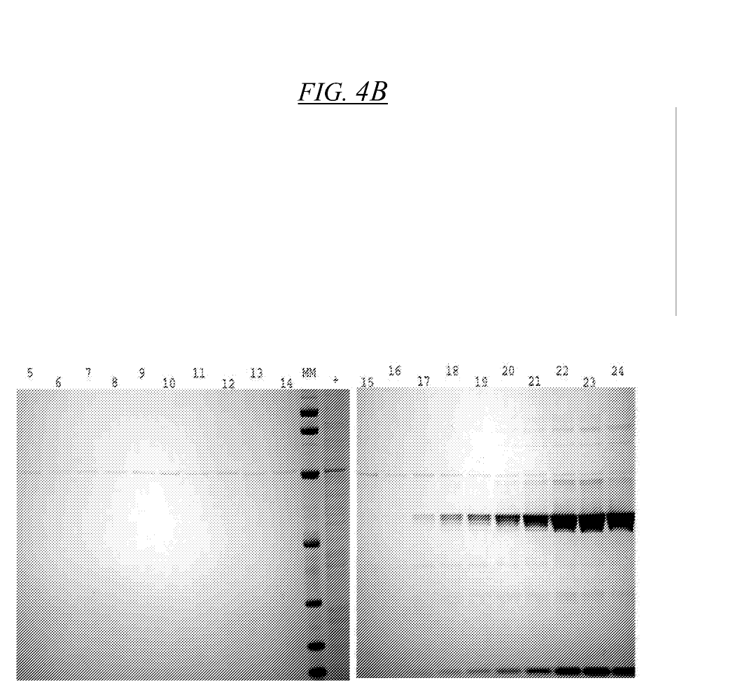

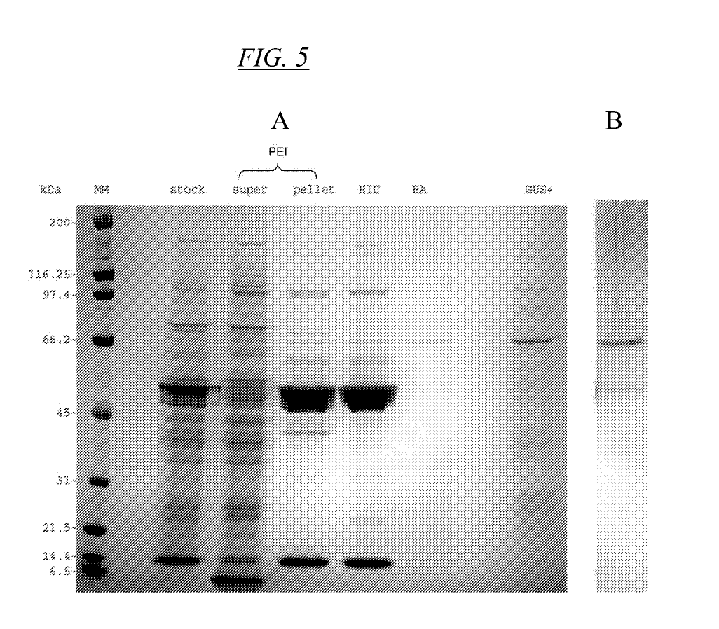

Further, FIG. 5 shows SDS-PAGE analysis of the entire purification scheme, stained with Bio-Safe Coomassie stain (Panel A) or silver stain (Panel B). As can be seen, the final product pooled from 16 fractions after HAC corresponds to approximately 40% of the initial rGUS activity and is nearly homogeneous after staining with silver stain (Panel B). These results are quite remarkable when considering the fact that no affinity purification methods were used, keeping the overall scheme general for use with a wide range of acidic proteins expressed in tobacco or other leafy crops. In addition, all of the steps incorporated in this process should be scalable for large-scale commercial protein purification.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only.

### REFERENCES

Balasubramaniam D, Wilkinson C, Van Cott K, Zhang C. 2003. Tobacco protein separation by aqueous two-phase extraction. J Chromatogr A 989(1): 119-29.

Beachy R N, Fitchen J H, Hem M B. 1996. Use of plant viruses for delivery of vaccine epitopes. Engineering Plants for Commercial Products and Applications 792:43-49.

Cheryan M. 1980. Phytic acid interactions in food systems. Crit Rev Food Sci Nutr 13(4):297-335.

Cramer C L, Boothe J G, Oishi K K. 1999. Transgenic plants for therapeutic proteins: linking upstream and downstream strategies. Curr Top Microbiol Immunol 240:95-118.

Daniell H, Streatfield S J, Wycoff K. 2001. Medical molecular farming: production of antibodies, biopharmaceuticals and edible vaccines in plants. Trends Plant Sci 6(5):2 19-26.

Davis D L, Nielson M T. 1999. Tobacco: Production, Chemistry and Technology. Davis D L, Nielson M T, editors. Oxford: Blackwell Science. 3 p.

Desai U A, Sur G, Daunert S, Babbitt R, Li Q. 2002. Expression and affinity purification of recombinant proteins from plants. Protein Expr Purif 25(1): 195-202.

Discipio R G, Davie E W. 1979. Characterization of Protein S, a Gamma-Carboxyglutamic Acid Containing Protein from Bovine and Human-Plasma. Biochemistry 18(5):899-904.

Fernandez-San Millan A, Mingo-Castel A, Miller M, Daniell H. 2003. A chloroplast transgenic approach to hyper-express and purify Human Serum Albumin, a protein highly susceptible to proteolytic degradation. Plant Biotechnology Journal 1(2):71-79.

Ganz P R, Sardana R, Dudani A K, Tackaberry B, Sauder C, Altosaar I. 1996. Expression of human blood proteins in transgenic plants: the cytokine GM-CSF as a model protein. In: Owen M R L, Pen J, editors. Transgenic plants: a production system for industrial and pharmaceutical proteins. London, UK: John Wiley & Sons. p 281-287.

Garger S J, Holtz R B, McCulloch M J, Turpen T H. 2000. Process for isolating and purifying viruses, soluble proteins, and peptides from plant sources. U.S. Pat. No. 6,033,895.

Goddijn O J M, Pen J. 1995. Plants as Bioreactors. Trends in Biotechnology 13(9):379-387.

Hamamoto H, Sugiyama Y, Nakagawa N, Hashida E, Matsunaga Y, Takemoto S, Watanabe Y, Okada Y. 1993. A new tobacco mosaic virus vector and its use for the systemic production of angiotensin-I-converting enzyme inhibitor in transgenio tobacco and tomato. Biotechnology (NY) 11(8):930-2.

Haq T A, Mason H S, Clements J D, Arntzen C J. 1995. Oral Immunization with a Recombinant Bacterial-Antigen Produced in Transgenic Plants. Science 268(521 1):714-716.

Holler C, Vaughan D, Zhang C. 2007. Polyethyleneimine precipitation versus anion exchange chromatography in fractionating recombinant beta-glucuronidase from transgenic tobacco extract. J Chromatogr A 1142(1):98-105.

Jefferson R A, Wilson K J. 1991. The GUS gene fusion system. In: Gelvin S B, Schilperoort R A, Venna D P S, editors. Plant Molecular Biology Manual. Dordrecht, The Netherlands: Kiuwer Academic. p 1-33.

Jervis L, Pierpoint W S. 1989. Purification Technologies for Plant-Proteins. Journal of Biotechnology 11(2-3): 161-198.

Kisiel W, Davie E W. 1981. Protein C. Methods Enzymol 80:320-332.

Kusnadi A R, Evangelista R L, Hood B E, Howard J A, Nikolov Z L. 1998a. Processing of transgenic corn seed and its effect on the recovery of recombinant betaglucuronidase. Biotechnol Bioeng 60(1):44-52.

Kusnadi A R, Hood E E, Witcher D R., Howard J A, Nikolov Z L. 1998b. Production and purification of two recombinant proteins from transgenic corn. Biotechnol Prog 14(1):149-55.

Lige B, Ma S W, Zhao D L, van Huystee R B. 1998. Cationic peanut peroxidase: Expression and characterization in transgenic tobacco and purification of the histidine-tagged protein. Plant Science 136(2):159-168.

Mason H S, Ball J M, Shi J J, Jiang X, Estes M K, Arntzen C J. 1996. Expression of Norwalk virus capsid protein in transgenic tobacco and potato and its oral immunogenicity in mice. Proceedings of the National Academy of Sciences of the United States of America 93(11):5335-5340.

Mejare M, Lilius G, Bulow L. 1998. Evaluation of genetically attached histidine affinity tails for purification of lactate dehydrogenase from transgenic tobacco. Plant Science 134(1):103-114.

Menkhaus T J, Bai Y, Zhang C, Nikolov Z L, Glatz C E. 2004. Considerations for the recovery of recombinant proteins from plants. Biotechnol Prog 20(4):1001-14.

Nikolov Z L, Woodard S L. 2004. Downstream processing of recombinant proteins from transgenic feedstock. Curr Opin Biotechnol 15(5):479-86.

Sebnke P C, Ferl R J. 1999. Processing of preproricin in transgenic tobacco. Protein Expr Purif 15(2):188-95.

Shewry P R, Fido Ri. 1996. Protein extraction from plant tissues. In: Doonan 5, editor. Methods Mol Biol. Totawa, N.J.: Humana Press. p 23-9.

Sijmons P C, Dekker B M, Schra.mmeijer B, Verwoerd T C, van den Elzen P J, Hoekema A.1990. Production of correctly processed human serum albumin in transgenic plants. Biotechnology (NY) 8(3):217-21.

Stoger E, Sack M, Fischer R, Christou P. 2002a. Plantibodies: applications, advantages and bottlenecks. Curr Opin Biotechnol 13(2):161-6.

Stoger E, Sack M, Pemn Y, Vaquero C, Torres E, Twyman R M, Christou P, Fischer R. 2002b. Practical considerations for pharmaceutical antibody production in different crop systems. Molecular Breeding 9(3):149-158.

1. A process for purification of a protein from biological material of a cell, said process comprising:
contacting the protein with a polyelectrolyte;
contacting the protein with a hydrophobic interaction resin; and
contacting the protein with hydroxyapatite.

2. The process of claim 1, wherein the polyelectrolyte comprises polyethyleneimine.

3. The process of claim 1, wherein contacting of the protein with a polyelectrolyte causes the protein to precipitate.

4. The process of claim 1, wherein the hydrophobic resin is phenyl sepharose.

5. The process of claim 1, wherein contacting the protein with a hydrophobic interaction resin comprises applying the protein to a hydrophobic interaction resin packed into a chromatography column to form a resin-protein complex.

6. The process of claim 5, wherein the protein is released from the resin by exposing the resin-protein complex to a solution that causes the protein to disassociate from the resin.

7. The process of claim 1, wherein contacting the protein with hydroxyapatite comprises applying the protein to hydroxyapatite packed into a chromatography column to form a hydroxyapatite-protein complex.

8. The process of claim 7, wherein the protein is released from the hydroxyapatite by exposing the resin-protein complex to a solution that causes the protein to disassociate from the hydroxyapatite.

9. The process of claim 1, further comprising one or more centrifugation steps.

10. The process of claim 1, further comprising expressing the protein in the cell.

11. The process of claim 10, wherein the protein is a recombinant protein.

12. The process of claim 11, wherein the cell is a plant cell.

13. The process of claim 11, wherein the plant is a leafy crop.

14. The process of claim 11, wherein the cell is a cell of a transgenic tobacco plant.

15. The process of claim 10, further comprising lysing the cell and homogenizing the lysate.

16. The process of claim 15, further comprising centrifuging the lysate.

17. The process of claim 15, further comprising filtering the lysate.

18. A process for purifying a protein from a tobacco cell, said method comprising:
lysing the tobacco cell;
homogenizing the lysate;
centrifuging the lysate and retaining the soluble fraction;
filtering the soluble fraction to provide a protein-containing sample;
precipitating the protein from the sample by contacting it with one or more polyelectrolytes;
binding the protein to a hydrophobic interaction resin, then releasing the protein from the resin; and
binding the protein to hydroxyapatite then releasing the protein from the hydroxyapatite.

19. The method of claim 18, wherein contacting the protein with one or more polyelectrolytes comprises:
contacting the two substances to form a mixture, sonicating the mixture, and performing two centrifugation steps to precipitate the protein from the mixture.

20. The method of claim 18, further comprising concentrating or diluting the protein prior to binding the protein to hydroxyapatite.

21. The process of claim 18, wherein binding the protein to a hydrophobic interaction resin comprises performing hydrophobic interaction column chromatography using phenyl sepharose FF low sub.

22. The process of claim 18, wherein binding the protein to hydroxyapatite comprises performing column chromatography of the protein using ceramic hydroxyapatite as the column matrix.
